SEQUENCE LISTING (9 sequences)

SEQ ID NO: 1 - DNA, 17 bp, Saccharomyces cerevisiae
gcgggtgacagccctcc

SEQ ID NO: 2 - DNA, 40 bp, Artificial
  Primers for Flp recombinase of S. cerevisiae 2 micron plasmid
cgcggattcaattaattatgccacaatttggtatattatg

SEQ ID NO: 3 - DNA, 31 bp, Artificial
  Primers for Flp recombinase of S. cerevisiae 2 micron plasmid
ccactcgagttatatgcgtctatttatgtag

SEQ ID NO: 4 - DNA, 104 bp, Artificial
  Artificial sequence from Saccharomyces cerevisiae GAL4 gene (in part)
gcgggtgacagccctccgcgggtgacagccctccgcgggtgacagccctccgcgggtgacagccctccgcgggtgacagccctccgcgggtgacagccctccgt

SEQ ID NO: 5 - DNA, 112 bp, Artificial
  Artificial sequence from Saccharomyces cerevisiae GAL4 gene (in part)
ctagacggagggctgtcacccgcggagggctgtcacccgcggagggctgtcacccgcggagggctgtcacccgcggagggctgtcacccgcggagggctgtcacccgctgca

SEQ ID NO: 6 - DNA, 60 bp, Artificial
  Artificial sequence from cauliflower mosaic virus (in part)
ctagagcgaagacccttcctctatataaggaagttcatttcatttggagaggacacgctg

SEQ ID NO: 7 - DNA, 59 bp, Artificial
  Artificial sequence from cauliflower mosaic virus (in part)
gatccagcgtgtcctctccaaatgaaatgaacttccttatatagaggaagggtcttgct

SEQ ID NO: 8 - DNA, 33 bp, Artificial
  Artificial sequence - primer for amplifying GAL4-vpI6
ccaggatccttaattaatgaagctcctgtcctc

SEQ ID NO: 9 - DNA, 26 bp, Artificial
  Artificial sequence - primer for amplifying GAL4-VP1
acgcgtcgacagatctacccaccgta

(19) United States
(12) Patent Application Publication
(10) Pub. No.: US 2019/0048358 A1
(43) Pub. Date: Feb. 14, 2019

(54) Expression of Foreign Sequences in Plants Using Trans-Activation System

(71) Applicant: iBio Inc., New York, NY (US)

(72) Inventors: Vidadi Yusibov, Havertown, PA (US); Vadim Mett, Elma, NY (US); Marina Skarjinskaia, New York, NY (US); Anna Hull, New York, NY (US)

(21) Appl. No.: 15/946,672

(22) Filed: Apr. 5, 2018

Related U.S. Application Data
(63) Continuation of application No. 10/832,603, filed on Apr. 26, 2004, now abandoned.
Application No. 10/832,603 is a continuation-in-part of application No. PCT/US2003/035869, filed on Nov. 6, 2003.
(60) Provisional application No. 60/424,275, filed on Nov. 6, 2002.
(60) Provisional application No. 60/465,474, filed on Apr. 25, 2003.

(51) Int. Cl. C12N 15/82

(52) U.S. Cl. CPC C12N 15/8216

(57) ABSTRACT

A transactivation system and method for producing a foreign polypeptide of interest in cells of a host plant is disclosed. The transactivation system has two components. It has genetically transformed cells of the host plant having integrated in their nuclear genome, an inactive or silenced foreign nucleic acid sequence, which is capable of encoding, upon its activation, the foreign polypeptide of interest; and a recombinant RNA viral vector capable of infecting the cells of the host plant and encoding therein a factor for activating or facilitating the expression of inactive or silenced foreign nucleic acid sequence.

### CROSS-REFERENCE TO RELATED APPLICATIONS

This application is a continuation of U.S. Ser. No. 10/832,603, filed Apr. 26, 2004, which is a continuation-in-part of international application PCT/US2003/035869, filed Nov. 6, 2003, which applications are hereby incorporated by reference in their entirety.

### FIELD OF THE INVENTION

The present invention is directed to the field of protein expression and Molecular Biology where plants are used as units for expression and production of foreign proteins or nucleotide sequences. The new plant-based production system allows controlled expression of foreign sequences in wide range of plants (monocot and divots) at different growth stages (sprouts, leaf stage, flowering, seed formation and maturation) in different organs (roots, stem, leaves, flowers, seed pods, seed coat, seeds). Specifically, methods are provided for producing transgenic plants with inactive transgenes and activation of the inactive transgenes using viral vectors.

### BACKGROUND OF THE INVENTION

The capability of single plant cell to regenerate and give rise to whole plant with all genetic features of the parent and ii) transfer of foreign genes into a plant genome by a plant-infecting bacterium, Agrobacterium tumefaciens (A. tumefaciens) enabled workers in this field to develop new procedures for crop improvement and stable expression of foreign proteins in plants.

In addition to transgenic plants, with the advances made in molecular plant virology, plant viruses have also emerged as promising tools. Plant viruses have features that range from detrimental to potentially beneficial. The substantial crop losses world-wide due to viral infections have prompted the molecular plant virologists to develop genetic systems that allow manipulation of the virus for management of plant diseases. These genetic systems have also led to the use of viruses as tools, since small plus-sense single-stranded RNA viruses that commonly infect higher plants can be used rapidly amplify virus-related RNAs and produce large amounts of protein.

During the last decade, commercial and academic interest in plants as heterologous expression system has significantly increased, especially for the production of biomedically important proteins. Plants have several advantages for producing therapeutic proteins, including lack of contamination with mammalian pathogens; relative ease of genetic manipulation; eukaryotic protein modification machinery; and economical production. Plants might also provide an ideal vehicle for oral delivery of vaccine antigens, since unlike mammalian cells, plant cells are enveloped with a thick cell wall consisting mainly of cellulose and sugars that may provide protection to the antigen inside against degradation in the gastrointestinal tract.

Both transgenic plants and engineered plant viruses have been used in producing foreign proteins in plant. In the early 1990, transgenic plant technology moved to a new arena as a heterologous expression system for antigens from mammalian pathogens. Since then, a variety of medically important antigens have been expressed in transgenic plants, including hepatitis B surface antigen (HBsAg) E. coli heat-labile enterotoxin, rabies virus glycoprotein, and Norwalk virus capsid protein.

Plants can also efficiently express and assemble functional immunoglobulins, including relatively complex secretory IgA (slgA) molecules composed of four separate polypeptide chains. Again, this points to the potential of transgenic plants in the bioproduction of complex therapeutic proteins. However, technical challenges remain that must be overcome before plant-based production of therapeutic proteins gains widespread acceptance in the commercial arena. Optimization of protein production levels, an important requirement to any heterologous expression system, is one of these challenges. At present, direct expression of recombinant proteins in transgenic plants does not always satisfy the requirement for high levels of protein expression. Plants have an excellent mechanism for turning down or silencing foreign gene expression, particularly when expression is constitutive. It is generally accepted that the duration of foreign gene expression is one of the critical factors that negatively affect accumulation of high quantities of target proteins. The discovery of inducible promoters somewhat circumvents this problem. Thus, there is considerable interest both in basic plant biology and in biotechnological applications, in the use of inducible systems for the expression of target genes introduced into plants. Compared to constitutive promoters, inducible promoters offer numerous advantages and potentials. In particular, inducible promoters allow for the generation of transgenic plants carrying a transgene whose expression at high level is detrimental or even lethal to the host plants. It is also considered that inducible gene expression will be much less sensitive to post-transcriptional gene silencing.

A number of inducible promoters that may allow control over the expression of target genes in transgenic plants have been described. Based on their specificity to a particular class of inducers these promoters could be divided into three groups: i) promoters that are induced at different developmental stages (flowering, senescence, etc.) in different organs (roots, flowers, seeds, etc.), ii) promoters that respond to particular environmental signals (heat-shock, nutritional status, pathogen attack or mechanical wounding), iii) promoters that are induced by chemicals of non-plant origin (tetracycline-, glucocorticoid-, ecdysteroid-, copper- and ethanol-inducible promoters). The latter generally utilize non-plant transcription factors that require chemical inducers for activation. Compared to the first two groups of promoters, chemical-inducible systems have much greater potential for a strict temporal and spatial control of the expression of the target gene expression in transgenic plants. Unfortunately, current inducible plant expression systems have some shortcomings, including leaky promoters or commercially unfeasible manufacturing conditions.

An alternative system for the expression of foreign proteins in plants is based on plant virus vectors. Although plant virus vector-based expression systems have a number of advantages (time, efficient engineering and production, level of target protein expression, environmental safety, etc.) compared to that of transgenic plants, they have some limitations as well. For example, the stability and systemic movement of the recombinant virus may be affected by the size of the target gene. Virus-based vectors are probably less applicable in projects that require coordinated expression of multi-subunit proteins, such as antibodies and enzyme complexes.

Accordingly, there is a need for creating better vectors and/or systems that are more suitable for commercial production of proteins in a controlled manner so that it does not create an environmental hazard yet provides for high levels of target protein or polypeptide accumulation in cells regardless of size and complexity of the proteins or polypeptides, the stability of engineered vectors, size and complexity of proteins that can be optimally produced in plants are some of the desirable properties that need to be realized. In addition, the vectors and/or systems must be environmentally friendly and conforming to the regulatory guidelines.

### SUMMARY OF THE INVENTION

The present invention provides for creating vectors and/or systems that are environmentally friendly and are suitable for commercial production of proteins or polypeptides in a controlled manner regardless of size and complexity of the proteins or polypeptides. In general aspects the present invention provides a solution by combining environmentally safe transgenic plants and plant viruses to create a transactivation system for production of target proteins in plants.

Specifically, in one aspect of the invention, a transactivation system for producing a foreign polypeptide of interest in cells of a host plant is provided. It has two components: (i) a transgenic plant and a recombinant viral vector. More specifically, the system has genetically transformed cells of the host plant having integrated in their nuclear genome, an inactive or silenced foreign nucleic acid sequence, which is capable of encoding, upon its activation, the foreign polypeptide of interest, and a recombinant RNA viral vector capable of infecting the cells of the host plant and encoding therein a factor for activating or facilitating the expression of inactive or silenced foreign nucleic acid sequence. The recombinant RNA viral vector has a recombinant genomic component of a class of virus such as TMV (tobamovirus), an alfalfa mosaic virus, an ilarvirus, a cucumovirus or a closterovirus. The recombinant RNA viral vector can be a heterologous viral vector, i.e., the genomic component has nucleic acid sequences from more than one class of virus. The host plant is a dicotyledon or a monocotyledon.

The foreign nucleic acid sequence integrated into the host genome is part of a transgenic DNA and has at its upstream end (i.e., in the direction of the 5′ end relative to the foreign nucleic acid sequence) a viral replicon responsive to a replication factor. The replication factor can be encoded by the recombinant RNA viral vector used for infecting the plant cells or by the host plant in which case the host plant is transgenic for expressing the replication factor (e.g. Rep transgenic tobacco known in the art). The foreign nucleic acid sequence is inactive or silenced due to a blocking sequence (e.g., a staffer sequence flanked on each side by an FRT site in a 5′ to 3′ orientation) between the foreign nucleic acid sequence and regulatory sequences in place for driving the expression of the foreign nucleic acid sequence. The foreign nucleic acid sequence can also be inactive due to lack of activity of a transcription factor activity within the plant cells. The nuclear genome of these cells has a DNA sequence to which the transcription factor can bind and activate the expression of inactive foreign nucleic acid sequence within the cells.

In an embodiment, the foreign nucleic acid sequence encodes an antigen of interest such as G protein of Respiratory Syncytial Virus, enterotoxin, rabies virus glycoprotein, rabies virus nucleoprotein, hepatitis B surface antigen, Norwalk virus capsid protein, colorectal cancer antigen and gastrointestinal cancer antigen.

In another embodiment, the foreign nucleic acid sequences encode biopharmaceuticals such as erythropoietins, interferons, insulins, monoclonal antibodies, blood factors, Colony Stimulating Factors, Growth Hormones, interleukins, Growth Factors and Vaccines.

In another aspect of the invention, a method for producing a foreign polypeptide in cells of a host plant is provided. It includes, among other steps, a step of generating a transgenic plant so that nuclear genome of cells of the transgenic plant has a transgenic DNA comprising an inactive or silenced foreign nucleic acid sequence, which is capable of encoding, upon its activation, the foreign polypeptide, a step of infecting the transgenic plant cells with a recombinant RNA viral vector so that it replicates and transiently expresses therein a factor for activating or facilitating the expression of the inactive or silenced foreign nucleic acid sequence; and a step of growing the plant, where the foreign polypeptides are produced in said cells.

In yet another aspect of the invention, a method for producing a foreign polypeptide in plant or animal cells in a culture (a fermentation culture) is provided. It includes, among other things, generating transgenic cells so that nuclear genome of those cells has a transgenic DNA having an inactive or silenced foreign nucleic acid sequence, which is capable of encoding, upon its activation, the foreign polypeptide, infecting the transgenic cells with a recombinant RNA viral vector so that it replicates and transiently expresses therein a factor for activating or facilitating the expression of the inactive or silenced foreign nucleic acid sequence, and growing the cells in a suitable culture medium, wherein the foreign polypeptides of interest are produced in the cells. The transgenic plant cells are cell suspensions or cells in a tissue selected from the group consisting of: root, shoot, flower and fruit.

### BRIEF DESCRIPTION OF THE DRAWINGS

FIG. 1 shows an embodiment of a transgenic DNA construct (A) containing target gene (TG) that is separated from the promoter by :FRT-Stuffer-FRT and a viral vector (B) with an FLP activator gene for expression in plants. MP is movement protein.

FIG. 2 shows another embodiment of an Agrobacterial DNA construct (A) that contains target gene (TG) and a viral vector (B) with an activator gene for expression in plants. The TG in this construct is cloned in the background of plant virus sequences to enhance the target expression. The background virus sequences could be a full-length self-replicating molecule (for example tobacco mosaic virus or potato virus X) or molecule that has recognition sequences and can be trans-replicated by the B.

FIG. 3 shows another embodiment of an Agrobacterial DNA construct (A) that contain two target gene (TG1 and TG2) in tandem that can be activated by: i) FLP recombinase and ii) Gal4 transcription factor and viral vectors that contain activator genes (B and C) FLP recombinase and Gal4 transcription factor, respectively, for expression in plants.

FIG. 4 shows another preferred embodiment of a transgenic DNA construct with target gene (TG) and a viral vector with an activator gene for expression in plants.

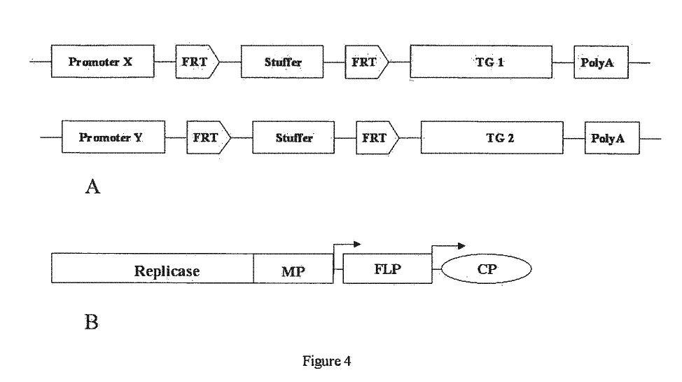

FIG. 5 shows another preferred embodiment of a transgenic DNA construct (A) with target genes (TG 1 and TG2 each having viral replicon sequences to its upstream end) and viral vectors (B and C) with each with an activator gene for expression in plants.

FIG. 6 shows photographs depicting the expression of GUS in Nicotiana benthamiana plants infected with virus vectors producing FLP recombinase. FLP recombinase was cloned into TMV based vectors, AvFLP, D4FLP and D40ASFLP. Here wild type means uninfected control.

FIG. 7 shows photographs depicting the expression of GUS in transgenic plants: FRT+/AgroFLP-transgenic plants containing GUS silenced with FRT were agroinfiltrated with agrobacterium containing FLP+/AgroFRT transgenic plants containing FLP were agroinfiltrated with construct containing FRT. No GUS activity was observed when FRT/FRT or FLP/FLP constructs were used.

FIG. 8 shows photographs depicting the local and systemic expression of GUS in transgenic Nicotiana benthamiana plants after infection with virus vector D4-FLP producing FLP recombinase.

FIG. 9 shows systemic expression of GUS in transgenic N. benthamiana plants infected with viral vector containing FLY transactivator (FLP recombinase)

FIG. 10 shows a schematic representation of viral vector construct containing GAL4-VP16 element, Arrow indicates the site of subgenomic promoter and the wavy line indicates the 3′ non-coding region.

FIG. 11 shows a schematic representation of agrobacterial construct containing target gene downstream of DNA binding and activation domains. The inducible system consists of two components; 1, a gene of interest placed downstream of six copies of the GAL4 upstream regulatory sequence (UAS) followed by a minimal promoter (TATA), and 2, a VP16-GAL4 fusion functioning as a transactivator to activate expression of the target gene.

FIG. 12 shows the expression of GUS as a result of trans-activation in plant producing GAL4-VP16.

FIG. 13A shows trans-activation of GUS and during co-infiltration of N. benthamiana plants expressing GAL4-VP16.

FIG. 13B shows Western analysis of trans-activation of GUS and IA-2ic during Co-infiltration of N. benthamiana plants expressing GAL4-VP16.

FIG. 14 shows the expression of GUS in pBIGAL/GUS transgenic plants infected with GAL4VP16/D4. The abbreviation “dpi” is days post inoculation.

FIG. 15 shows Western analysis of expression of TA IgG in transient assays.

FIG. 16 shows data on Binding of plant produced 9F12 antibody to tetanus toxin in ELISA. Microplate wells were coated with 0.5 μg tetanus toxin and incubated with serial dilutions of the 9F12 (▴) or an unrelated (▪) antibody purified from plants by a protein A affinity chromatography. Data represent the average of three replicate assays.

### DETAILED DESCRIPTION OF THE INVENTION

The present invention provides vectors and methods for expression of foreign sequences (peptides, polypeptides, and RNA) in plants. Specifically, the present invention relates to vectors and methods for activation of silenced or inactive foreign nucleic acid sequence(s) or gene(s) of interest in plant and animal cells for production of peptides, polypeptides, and RNA in such cells. The vectors used for the activation of silenced or inactive sequence(s) are viral vectors.

The activation of silenced or inactive foreign nucleic acid sequence(s) or gene(s) in plant or animal cells is achieved, in trans, by a factor (e.g., a protein or polypeptide) encoded by a nucleic acid sequence located on the viral vector after the cells are infected with the viral vector. In other words, delivery of activator gene via infection with transient gene delivery viral vector into plant or animal cell activates and results in the expression of target sequence(s). Thus, in the present invention, the activation of silenced or inactive foreign nucleic acid sequence(s) or gene(s) in plant or animal cells is transactivation. It is transactivation because the factor(s) are encoded by nucleic acid sequences that are remotely located, i.e., on the viral vectors, and the factor(s) are free to migrate or diffuse through the cell to their sites of action.

Accordingly, to practice the present invention, two components are needed as part of the transactivation system. First component of the transactivation system disclosed herein is a genetically transformed cell or organism, preferably a plant of interest containing the foreign nucleic acid sequence(s) or gene(s) of interest. The second component of the transactivation system is a viral vector, preferably a plant viral vector, capable of infecting the genetically transformed cell or organism (i.e. a host cell or a host) and encoding a transacting factor once delivered into the host cell.

To create the first component of the transactivation system, a suitable host cell or a host is genetically transformed with a foreign nucleic acid sequence or a transgene or target gene (TG) of interest to produce a transgenic cell or an organism. The genetic transformation is stable, i.e., the transgene is integrated into the host genome and is inheritable from one generation to the next. Plant cells and plants are preferred as host cells or hosts. Agrobacterium-based transformation methods may be used to produce transgenic plants containing target gene(s). Several other methods for stable transformation of plants, such as gene gun or vacuum infiltration are known in the art.

The terms target gene, transgene and foreign nucleic acid sequence are used interchangeably herein and refer to a nucleic acid sequence introduced into a host cell or a host through a stable genetic transformation procedure, which nucleic acid sequence, if operably linked to a promoter, is capable of encoding a foreign polypeptide or protein of interest such as for example, vaccine antigen, antibodies, therapeutic polypeptides, reporter molecules (e.g., β-glucuronidase (GUS) reporter gene, GFP) etc.

The preferred foreign nucleic acid sequences are those encoding various foreign polypeptides of interest such as, for example, erythropoietins, interferons, insulins, monoclonal antibodies, blood factors, Colony Stimulating Factors, Growth Hormones, Interleukins, Growth Factors, Vaccines, miscellaneous polypeptide drugs such as calcitonin, Leuprolide and HCG etc. These proteins or polypeptides are currently being produced by means other than the transactivation in transgenic cells or organisms described herein and are being marketed under various trade names as biopharmaceuticals.

The target gene of interest may be fused with other sequences that facilitate transport across the cell membranes, tissues and/or systemic delivery. See, for example, U.S. Pat. No. 6,051,239 for sequences which can be fused to the target gene of interest. As part of creating the first component of the transactivation system, a nucleic acid construct is introduced into the plant cell or a plant via a genetic transformation procedure. The nucleic acid construct can be a circular construct such as a plasmid construct or a phagemid construct or cosmid vector or a linear nucleic acid construct including, but not limited to, PCR products. Regardless of the form, the nucleic acid construct introduced is a cassette (also referred to herein as a transfer cassette or an expression cassette) having elements such as promoter(s) and/or enhancer(s) elements besides target gene(s) or the desired coding sequence, among other things. Expression of the target gene, however, depends on transactivation provided by the second component of the invention described further below.

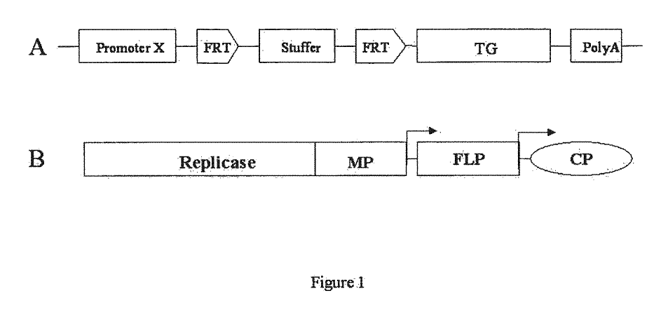

The transactivation system can be a recombinase based transactivation system or a transcription factor type (with activation and binding domains) based transactivation system. In the recombinase based transactivation system, the gene of interest (target gene or TG) is cloned into a transfer cassette (or a transformation plasmid) for integration into the plant genome and stable transformation. The target gene in the transformation plasmid is made non-functional by placing a blocking sequence between the promoter (and other regulatory sequences) for driving the expression of the target gene and the target gene. The resulting transfer cassette (or transgenic DNA) is said to have, among other things, the following structure: promoter-blocking sequence-TG. FIG. 1A shows a schematic representation of a transfer cassette in an embodiment of the present invention.

Promoters used in the present invention can be ubiquitous or constitutive (e.g., Cauliflower Mosaic Virus 35S promoter) or tissue specific promoters (e.g., potato protease inhibitor II (pin2) gene promoter, promoters from a number of nodule genes). A number of such promoters are known in the art. Inducible promoters that specifically respond to certain chemicals (copper etc.,) or heat-shock (HSP) are also contemplated. Numerous tissue specific and inducible promoters have been described from plants. The blocking sequence contains a selectable marker element or any other nucleic acid sequence (referred to herein as stiffer) flanked on each side by a recombinase target site (e.g., “FRT” site) with a defined 5′ to 3′ orientation. The FRT refers to a nucleic acid sequence at which the product of the FLP gene, i.e., FLP recombinase, can catalyze the site-specific recombination.

The selectable marker element or staffer is generally an open reading frame of a gene or alternatively of a length sufficient enough to prevent readthrough. When a suitable recombinase is provided by the second component of the transactivation system to the cells of the transgenic plant containing the transgenic DNA or expression cassette, the recombinase protein can bind to the two target sites on the transgenic DNA, join its two target sequences together and excise the DNA between them so that the target gene is attached to a promoter and/or an enhancer in operable linkage. The recombinase is provided in cells by a viral vector and the recombinase activates the expression of the target gene in cells where it is otherwise silenced or not usually expressed because of the blocking sequence.

It should be noted that the type of recombinase, which is provided to the plant cells in the present invention, would depend upon the recombination target sites in the transgenic DNA (or more specifically in the targeting cassette). For example, if FRT sites are utilized, the FLP recombinase is provided in the plant cells. Similarly, where lox sites are used, the Cre recombinase is provided in the plant cells. If the non-identical sites are used, for example both an FRT and a lox site, then both the FLP and Cre are provided in the plant cells.

The recombinases used herein are sequence-specific recombinases. These are enzymes that recognize and bind to a short nucleic acid site or a target sequence and catalyze the recombination events. A number of sequence-specific recombinases and their corresponding target sequences are known in the art. For example, the FLP recombinase protein and its target sequence, FRT, are well-characterized and known to one skilled in the art. Briefly, the FLP is a 48 kDa protein encoded by the plasmid of the yeast, Saccharomyces cerevisiae. The FLP recombinase function is to amplify the copy number of the plasmid in the yeast. The FLP recombinase mediates site-specific recombination between a pair of nucleotide sequences, FLP Recognition Targets (FRT's). The FRT is a site for the 48 kDa FLP recombinase. The FRT site is a three repeated DNA sequences of 13 bp each; two repeats in a direct orientation and one in an inverted to the other two. The repeats are separated by the 8 bp spacer region that determine the orientation of the FRT recombination site. Depending of the orientation of the FRT sites FLP-mediated. DNA excision or inversion occurs. FRT and FLP sequences can be either wild type or mutant sequences as long as they retain their ability to interact and catalyze the specific excision. Transposases and integrases and their recognition sequences may also be used.

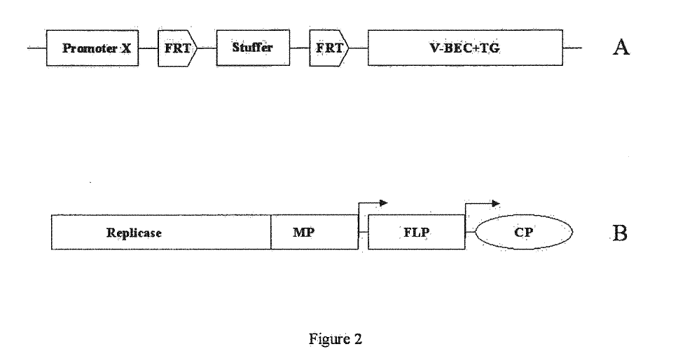

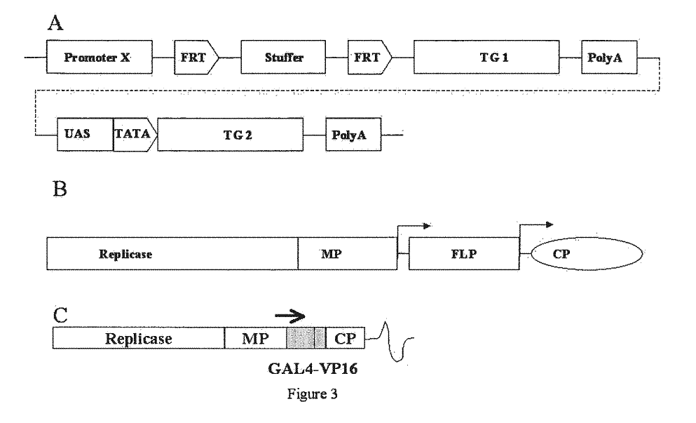

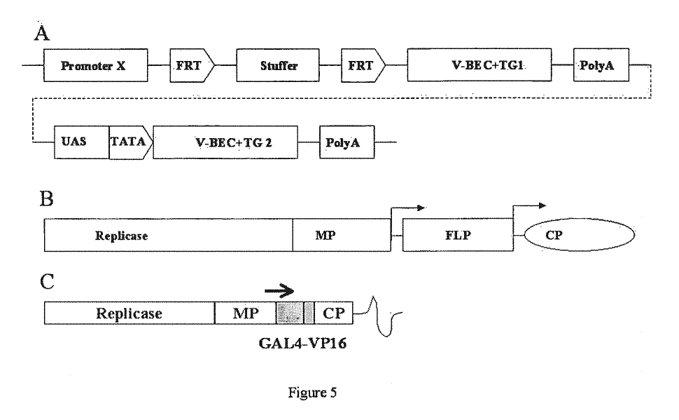

Shown in FIG. 2 is a transfer cassette according to another embodiment of the invention. In this embodiment, a viral replicon (e.g., V-BEC) is placed upstream of a target gene (FIG. 2A). See also FIG. 5A. The viral replicon is a viral nucleic acid sequence which allows for the extrachromosomal replication of a nucleic acid construct in a host cell expressing the appropriate replication factors. The replication factor may be provided by a viral vector or a transgenic plant carrying a replicase transgene. Such transgenic plants are known in the art. See, for example, PCT International Publication, WO 00/46350. The constructs of the present invention containing a viral origin of replication, once transcribed, replicate to a high copy number in cells that express the appropriate replication factors. The transfer cassette may contain more than one target gene each linked to a promoter and other elements. Each of the target genes may be transactivated by factors provided by a specific viral vector in a host cell. See, for example, FIGS. 3 and 5 where it is shown that target genes 1 and 2 are cloned sequentially in a stable transformation vector.

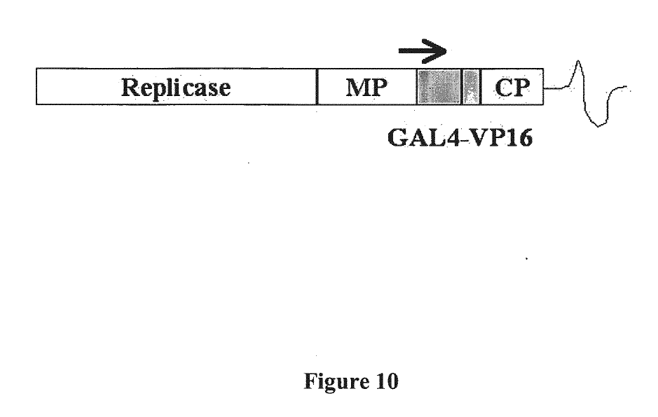

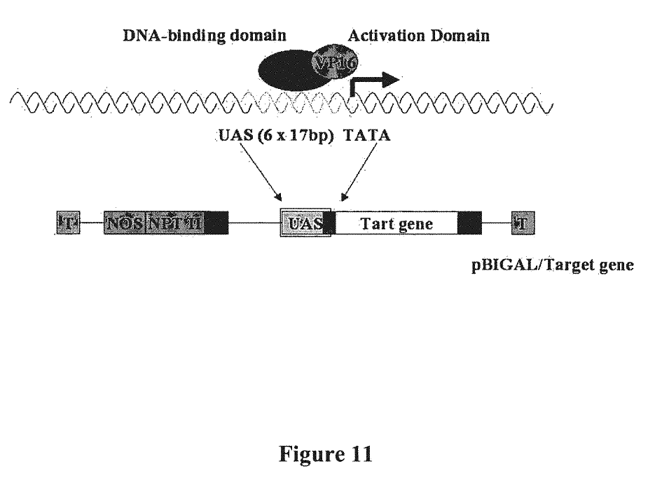

In the transcription factor type (for example with activation and binding domains) based transactivation system, the gene of interest (target gene or TG) is cloned into a transfer cassette (or a transformation plasmid) for integration into the host genome (animal or plant) and stable transformation. The target gene will only be expressed when a suitable transcription factor activity is available. This can happen when a fusion protein containing a DNA-binding domain and an activation domain interacts with certain regulatory sequences cloned into the transfer cassette that is integrated into the host genome. For example, FIGS. 10 and 11 show constructs for a transcription factor type based transactivation system, specifically the GAL4A-VP16 trans-activation system. It is based on the binding of the yeast GAL4-transcritpion factor to its upstream activating sequence UAS in combination with the strong transcriptional activator VP16 from Herpes Simplex Virus. The viral vector shown in FIG. 10 encodes GAL4-VP16 in plants. The transfer cassette shown in FIG. 11 has a gene of interest placed downstream of six copies of the GAL4 upstream regulatory sequence (UAS;) followed by a minimal promoter (TATA). The GAL4-VP16 fusion protein (as a transactivator) activates the expression of the target gene by binding to the sequences in the UAS.

Specifically, the expression system has: i) a transgene under the control of an artificial promoter containing binding sites for the Saccharomyces cerevisiae GAL4 transcription factor and a minimal TATA fragment, and ii) a hybrid GAL4-VP16 transcription factor (trans-activator) encoded by a virus-based vector. The target gene is not transcribed until the plants are inoculated with the recombinant virus expressing the trans-activator. The trans-gene is placed downstream of six repeats of the yeast GAL4 upstream activating sequence (UAS), followed by a minimal promoter sequence. The transgene activation takes place when a fusion-protein having the GAL4 DNA binding domain and VP16 transcription activating-domain is introduced into the plant via viral-vector mediated transient expression.

There are a number of trans-acting factors and the corresponding DNA sequences (i.e., cis-acting elements) known in the art. Spl transacting factor and the DNA sequence identical to or a variant of GGGCGG, TFIID transacting factor and the DNA sequence identical to or a variant of TATAAA, C/EBP transacting factor and the DNA sequence identical to or a variant of CCAAT, and ATF-1 transacting factor and the DNA sequence identical to or a variant of GTGACGTA are some examples that can be used in creating the two components of a transactivation system of the present invention.

Viral vectors are used to deliver factors for transactivation of inactive or silenced target genes in transgenic host cells or organisms.

The viral vectors used herein are of RNA type and do not integrate into host genome and the expression is extrachromosomal (transient or in the cytoplasm). Recombinant plant viruses are used in the case of transgenic plant cells or plants. The use of plant viral vectors for expression of recombinases in plants provides a means to have high levels of gene expression within a short time. The autonomously replicating viruses offer several advantages for use as gene delivery vehicles for transient expression of foreign genes, including their characteristic high levels of multiplication and transient gene expression. The recombinant viral vectors used in the present invention are also capable of infecting a suitable host plant and systemically transcribing or expressing foreign sequences or polypeptides in the host plant. Systemic infection or the ability to spread systemically of a virus is an ability of the virus to spread from cell to cell and from infected areas to uninfected distant areas of the infected plant, and to replicate and express in most of the cells of the plant. Thus this ability of plant viruses to spread to the rest of the plant and their rapid replication would aid in delivery of factors for transactivation throughout the plant and the consequent large scale production of polypeptides of interest in a short time.

Therefore, the invention requires construction of recombinant viral vectors by manipulating the genomic component of the wild-type viruses. Preferred viruses are RNA containing plant viruses. Although many plant viruses have RNA genomes, it is well known that organization of genetic information differs among groups. Thus, a virus can be a mono-, bi-, tri-partite virus. “Genome” refers to the total genetic material of the virus. “RNA genome” states that as present in virions (virus particles), the genome is in RNA form.

Some of the viruses which meet this requirement, and are therefore suitable, include Alfalfa Mosaic Virus (Al MV), ilarviruses, cucurnoviruses such as Cucumber Green Mottle Mosaic virus (CGMMV), closteroviruses or tobamaviruses (tobacco mosaic virus group) such as Tobacco Mosaic virus (TMV), Tobacco Etch Virus (TEV), Cowpea Mosaic virus (CMV), and viruses from the brume mosaic virus group such as Brome Mosaic virus (BMV), broad bean mottle virus and cowpea chlorotic mottle virus. Additional suitable viruses include Rice Necrosis virus (RNV), and geminiviruses such as tomato golden mosaic virus (TGMV), Cassava latent virus (CLV) and maize streak virus (MSV). Each of these groups of suitable viruses are well characterized and are well known to the skilled artisans in the field. A number of recombinant viral vectors have been used by those skilled in the art to transiently express various polypeptides in plants. See, for example, U.S. Pat. Nos. 5,316,931 and 6,042,832; and PCT International Publication, WO 00/46350, WO 96/12028 and WO 00/25574, the contents of which are incorporated herein by reference. Thus, the methods already known in the art can be used as a guidance to develop recombinant viral vectors of the present invention to deliver transacting factors.

The recombinant viral vector used in the present invention can be heterologous virus vectors. The heterologous virus vectors as referred to herein are those having a recombinant genomic component of a given class of virus (for example TMV) with a movement protein encoding nucleic acid sequence of the given class of virus but coat protein (either a full-length or truncated but functional) nucleic acid sequence of a different class of virus (for example AIMV) in place of the native coat protein nucleic acid sequence of the given class of virus. Likewise, native movement protein nucleic acid sequence instead of the coat protein sequence is replaced by heterologous (i.e. not native) movement protein from another class of virus. Shown in Figures SB and SC are schematics of heterologous vectors. For example, a TMV genomic component having an AIMV coat protein is one such heterologous vector. Similarly, an AIMV genomic component having a TMV coat protein is another such heterologous vector. The vectors are designed such that these vectors, upon infection, are capable of replicating in the host cell and transiently activating genes of interest in transgenic plants. Such vectors are known in the art, for example, as described in PCT International Publication, WO 00/46350.

In addition to the genomic elements necessary for infection, replication, movement and spread of the viral vectors, the vectors contain sequences encoding a recombinase (e.g., FLP) or other factor (e.g., GAL4-VP16).

In accordance with the present invention, the host plants included within the scope of the present invention are all species of higher and lower plants of the Plant Kingdom. Mature plants, seedlings, and seeds are included in the scope of the invention. A mature plant includes a plant at any stage in development beyond the seedling. A seedling is a very young, immature plant in the early stages of development. Specifically, plants that can be used as hosts to produce foreign sequences and polypeptides include and are not limited to Angiosperms, Bryophytes such as Hepaticae (liverworts) and Musci (mosses); Pteridophytes such as ferns, horsetails, and lycopods; Gymnosperms such as conifers, cycads, Ginkgo, and Gnetales; and Algae including Chlorophyceae, Phaeophpyceae, Rhodophyceae, Myxophyceae, Xanthophyceae, and Euglenophyceae.

Host plants used for transactivation of genes can be grown either in vivo and/or in vitro depending on the type of the selected plant and the geographic location. It is important that the selected plant is amenable to cultivation under the appropriate field conditions and/or in vitro conditions. The conditions for the growth of the plants are described in various basic books on botany, Agronomy, Taxonomy and Plant Tissue Culture, and are known to a skilled artisan in these fields.

Among angiosperms, the use of crop and/or crop-related members of the families are particularly contemplated. The plant members used in the present methods also include interspecific and/or intergeneric hybrids, mutagenized and/or genetically engineered plants. These families include and not limited to Leguminosae (Fabaceae) including pea, alfalfa, and soybean; Gramineae (Poaceae) including rice, corn, wheat; Solanaceae particularly of the genus Lycopersicon, particularly the species esculentum (tomato), the genus Solanum, particularly the species tuberosum (potato) and melongena (eggplant), the genus Capsicum, particularly the species anmum (pepper), tobacco, and the like; Umbelliferae, particularly of the genera Daucus, particularly the species carota (carrot) and Apium, particularly the species graveolens dulce, (celery) and the like; Rutaceae, particularly of the genera Citrus (oranges) and the like; Compositae, particularly the genus Lactuca, and the species sativa (lettuce), and the like and the family Cruciferae, particularly of the genera Brassica and Sinapis. Examples of “vegetative” crop members of the family Brassicaceae include, but are not limited to, digenomic tetraploids such as Brassica juncea (L.) Czem, (mustard), B. carinata Braun (ethopian mustard), and monogenomic diploids such as B. oleracea (L.) (tole crops), B. nigra (L.) Koch (black mustard), B. campestris (L.) (turnip rape) and Raphanus sativus (L.) (radish). Examples of “oil-seed” crop members of the family Brassicaceae include, but are not limited to, B. napus (L.) (rapeseed), B. campestris (L.), B. juncea (L.) Czem, and B. tournifertii and Sinapis alba (L.) (white mustard). Flax plants are also contemplated.

Particularly preferred host plants are those that can be infected by AIMV. For example, it is known in the art that alfalfa mosaic virus has full host range. Other species that are known to be susceptible to the virus are: Abelmoschus esculentus, Ageratum conyzoides, Amaranthus caudatus, Amaranthus retroflexus, Antirrhimum majus, Apium graveolens, Apium graveolens var. rapaceum, Arachis hypogaea, Astragalus glycyphyllos, Beta vulgaris, Brassica campestris ssp. rapa, Calendula officinalis, Capsicum annuum, Capsicumfrutescens, Caryopteris incana, Catharanthus roseus, Celosia argentea, Cheiranthus cheiri, Chenopodium album, Chenopodium amaranticol, Chenepodium murale, Chenopodium guinea, Cicer arieuinum, Cichium endiva,Ciandrum safivum, Crotalaria spectabilis, Cucumis melo, Cucumis sativus, Cucurbita pepo, Cyantepsis tetragonoloba, Daucus careta (var. saliva), Dianthus harbatus, Dianthus caryophyllus, Emilia sagittata, Fagopyrum esculentum, Glycine max, Gomphrena globosa, Helianthus annuus, Lablab purpureus, Lactuca saliva, Lathyrus odatus, Lens culinaris, Linum usitatissimum, Lupinus a/bus, Lycopersicon esculentum, Macroptillum lathyroides, Malva parvifla, Matthiola incana, Medicago hispida, Medicago saliva, Melilotus albus, Nicotiana bigelovii, Nicotiana clevelandii, Nocotiana debneyi, Nicotiana glutinosa Nicotiana megalosiphon, Nicotiana rustica, Nicotiana sylvestris, Nicotiana tabacum, Ocimum basilicum, Petunia x hybrida, Phaseolus lumatus, Phaseolus vulgaris, Philadelphus, Physalis flidana, Physalis peruviana, Phytolacca americana, Pisum sativum, Solanum demissum, Solanum melongena, Solanum nigrum, Solanum nodiflum, Solanum rostratum, Solanum tuberosum, Sonchus oleraceus, Spinacia oleracea, Ste/Zaria media. Tetragonia tetragonioides, Trifolium dubium, Tryblium hybridum, Trifolium incarnatum, Trifolium pratense, Trifolium repens, Trifolium subterraneum, Tropaeolum majus, Viburnum opulus, Viciafaba, Vigna radiata, Vigna unguiculata, Vigna unguiculata ssp, sesquipedalis, and Zinnia elegans.

An exemplary experimental design for the expression of GFP in transgenic plants through transactivation with recombinase produced in plants via infection with virus can be follows:

A plant virus vector (Av or AIMV) is engineered to express FLP recombinase. The gene for this protein is cloned under subgenomic promoter for coat protein, movement protein or artificial subgenomic promoter. The target gene is cloned into an agrobacterial vector and introduced into nuclear genome to obtain transgenic plants. The target gene is placed under a strong promoter (ubiquitin, dub35, super). However the expression is silenced by the introduction of NPT or stiffer sequence flanked by FRT (blocking sequence). Target gene is activated by removing the blocking sequences. There can be more than one target gene in a transfer cassette. The target gene(s) is (are) cloned into an agrobacterial vector and introduced into nuclear genome or chloroplast genome. These transformation procedures are well known in the art. Target gene is placed under strong promoter (ubiquitin, dub35, super). However, the expression is silenced by the introduction NPT or stuffer sequences flanked by recombinase recognition sites (e.g., FRT or lox) between the promoter and the TG. The target gene is activated by removing sequences between the promoter and the TG. There could be more than one target gene. The virus vector capable of expressing recombinase in plant cells and transgenic plants (nuclear or chloroplast) that are so made can readily be used to produce target proteins. Transgenic plants are infected with virus containing gene for recombinase.

Inoculation of plants; sprouts, leaves, roots, or stems is done using infectious RNA transcripts, infectious cDNA clones or pregenerated virus material. See, PCT International Publication, WO 00/46350 for guidance on infectious RNA transcripts and procedures for viral infection. Because the time span for target protein production according to the present invention is short (up to 15 days) the expression may not be affected by the gene silencing machinery within the host.

An exemplary experimental design for the GAL4-VP16 trans-activation system is as follows: The GAL.4-VP16 fusion protein is expressed from a viral vector. The GAL4-VPI6 gene is introduced into a viral vector under the control of a subgenomic promoter for coat protein, movement protein or an artificial subgenomic promoter. The target gene is cloned into an agrobacterial vector and introduced into the nuclear genome to obtain transgenic plants. The agrobacterial vector can be derived from the vector pBI121 or the like known in the art, in which the 35S CaMV or other promoter is replaced by 6 repeats of the 17 bp GAL4UAS (GCGGGTGACAGCCCTCC) (SEQ ID NO:1) followed by the minimal promoter sequence of 358 CaMV (−46 to +8 bp) or other. Transcription of the target gene can be activated by introduction of the GAL4-VP16 trans-activator.

Once the two components are ready, the transgenic plants can be inoculated with virus carrying the GAL4-VP 16 trans-activator to produce the target protein. Inoculation of plants can be made with infectious RNA transcripts, infectious cDNA clones or pre-generated virus material, which are well known in the art.

The transactivation system described herein can be adapted for polypeptide production in transgenic animal cells or non-human organisms (e.g., transgenic mammals, transgenic mouse, transgenic rabbit, transgenic rat, transgenic) containing the target gene of interest. Briefly, a suitable transformation procedure is used to genetically transform animal cells or organisms. Transfer cassettes can be accordingly designed for transformation by gene gun or electroporation or the like. The viral vectors can be, for example, adenoviral vectors capable of expressing trans acting factors (FLP or GAL4-VP16). Recombinant viral vectors capable of expressing various genes of interest are well known to one skilled in the art. See, for example, U.S. Pat. Nos. 6,297,357 and 6,596,698.

### EXAMPLES

The following examples further illustrate the present invention. The examples below are carried out using standard techniques, that are well known and routine to those of skill in the art, except where otherwise described in detail. The examples are offered by way of illustration and not by way of limitation.

### Example 1

### FLP Recombinase Mediated Transactivation of Inactive Transgenes in Cells

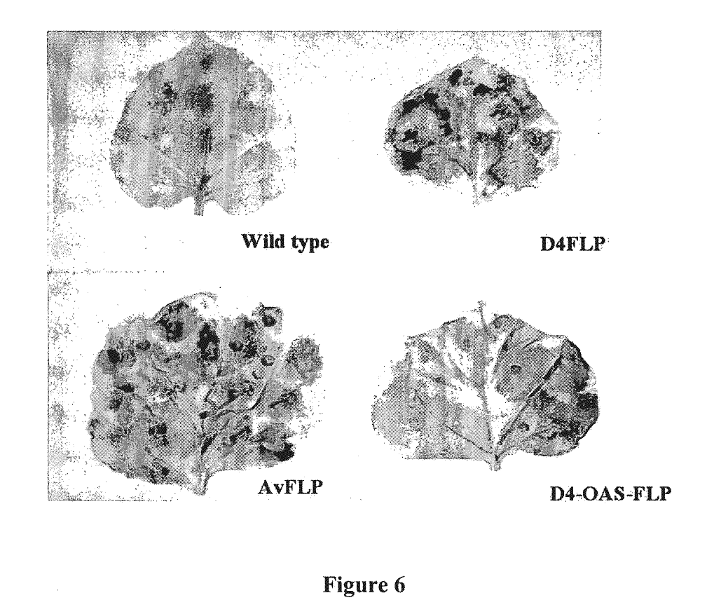

Construction of FLP encoding viral vector and activation of silenced GUS gene in plant cells were carried out as described in the paragraphs below. (For silencing or inactivation of the GUS gene in plant cells a transformation cassette in which the GUS gene was separated from CaMV 35S RNA promoter by the FRT flanked neo sequence or the stiffer sequence as schematically shown in FIG. 1A was used.
1. Engineering of virus constructs containing FLP recombinase. An open reading frame of yeast recombinase FLP is PCR-amplified from pJFLO plasmid (Luo H, Lyznik L A, Gidoni D, Hodges T K. 200( )FLP-mediated recombination for use in hybrid plant production. Plant J. 23:423-30) using 5′CGC GGA TTC AAT TAA TTA TGC CAC AAT TTG GTA TAT G3′ (SEQ ID NO:2) and 5′CCA CTC GAG TTA TAT GCG TCT ATT TAT GTA G3′ (SEQ ID NO:3) as 5′ and 3′ primers, respectively. BamHI and Pacl restriction sites at the 5′ and Xhol at the 3′ end, respectively were introduction for cloning into final vector. Resulting PCR fragment is digested with BamHI and Xhol and cloned in pBluescript. Recombinant plasmid are analyzed by restriction mapping and sequencing to confirm the coding sequences.
2. After sequence confirmation 1.3 kb Pacl-Xhol fragment that contained FLP recombinase open reading fragment (from 1) was cloned into a different TMV virus based expression vectors, including D4, Av, and 30B. The insert is confirmed by restriction digest analysis.
3. Plasmid DNA from recombinant virus constructs, containing FLP recombinase was used to synthesize in vitro transcripts using T7 RNA polymerase. The resulting RNA transcripts were used to inoculate leaves of reporter transgenic plants transformed with pFFG (Luo H, Lyznik L A, Gidoni D, Hodges T K. 2000 FLP-mediated recombination for use in hybrid plant production. Plant J. 23:423-30) containing GUS gene separated from CaMV 35S RNA promoter by the neo sequence flanked by FRT sites.
4. The inoculated leaves from (3) are sampled after a period of growth at different time points and the activity of GUS reporter enzyme is visualized using histochemical staining with X-gluc substrate. Blue staining indicates expression of the reporter as a result of excision of neo cassette by FLP recombinase (FIGS. 6, 7, 8, and 9).
5. The experiment is conducted as in (3 and 4) except for co-inoculation with in vitro transcript from AvA4 providing systemic movement function in trans. In this case blue staining in non-inoculated upper leaves indicates systemic phloem movement of the FLP-expressing RNA, which activates the expression of GUS gene as a result of excision of neo cassette.
   A: Expression of β-Glucuronidase Reporter Gene in Nicotiana Benthamiana Plant by Trance-Activation via Infection with Virus Vector Containing FLP Recombinase:

To demonstrate that the inducible plant expression system described above allows for tight controlled expression of a trans-gene that is placed downstream of stuffer flanked with FRT sequences the inventors introduced into plants, a viral vector (see FIG. 1 B) transiently expressing FLP activator and followed with agroinfiltration of construct containing silenced GUS. It is shown here that the target proteins can be expressed in a fully transient manner.

In brief Nicotiana benthamiana plants (at 6 leaf stage) were independently inoculated with in vitro transcripts of the viral vectors containing FLP (trans-activator) gene under the control of subgenomic promoter for mRNA of viral coat protein. Approximately five to seven days post-inoculation symptoms (curling of leaves) of virus infection could be observed.

Ten days post inoculation with in vitro transcripts of the viral vectors containing FLP the leaves were infiltrated with an agrobacterium suspension carrying GUS gene silenced by introducing stuffer flanked with FRT sequences. Strong β-glucuronidase activity was observed in all leaves which expressed the FLP trans-activator and were infiltrated with agrobacterium containing the β-glucuronidase target gene, while no β-glucuronidase activity was detectable in any of the control leaves (FIG. 6). In addition, no β-glucuronidase activity was observed in leaves that expressed FLP but did not receive β-glucuronidase gene during agro-infiltration.

This is a strong indication that the FLP/FRT trans-activation system allows for tight controlled expression of the desired target gene. The trans-activation system described in this document can be used for expression of both single subunit as well as multi-subunit complex target proteins, including antibodies that require simultaneous expression of two target genes.

### B: Expression of Target Gene in Transgenic Plants by Trans-Activation via Agroinfiltration:

A. To show that the system allows expression of target gene in transgenic plants, transgenic henthamiana plants containing either GUS gene silenced by stater or FLP recombinase were created.

Plant Transformation: Nicotiana benthamiana were transformed with pB1-based target constructs, containing target gene bordered with FRT elements or GAL4 transcription activator binding site as follows: The youngest, fully expanded leaf, was sterilized in 20% bleach containing 0.01% Tween for 7 min and then rinsed three times in sterile water. A 100 ml saturated culture of agrobacterium (strain GLA 4404) was resuspended in 20 ml MS media. The sterilized leaves were incubated in the agrosuspension for 5 to I 0 min. During the incubation the leaves were cut into one by one cm pieces. The leaf-pieces were co-cultivated in the dark at 21° C. on MS-2 plates (MS media with 20 g/l sucrose and 0.7% agar) for three days where after they were transferred to regeneration media (MS salts, 30 g/l sucrose, 0.7% agar, 0.2 mg/l NAA, 1 mg/l BA) containing ling 1 mg/l zeatin, 150 mg/l Km and 500 mg/l cefotaxime. When shoots appeared they were transferred to rooting media (Yi MS media containing 20% sucrose and 0.7% agar) with 250 mg/l cefotaxime. Plantlets with roots were transferred to soil and acclimated under plastic wrap for 4-7 days.

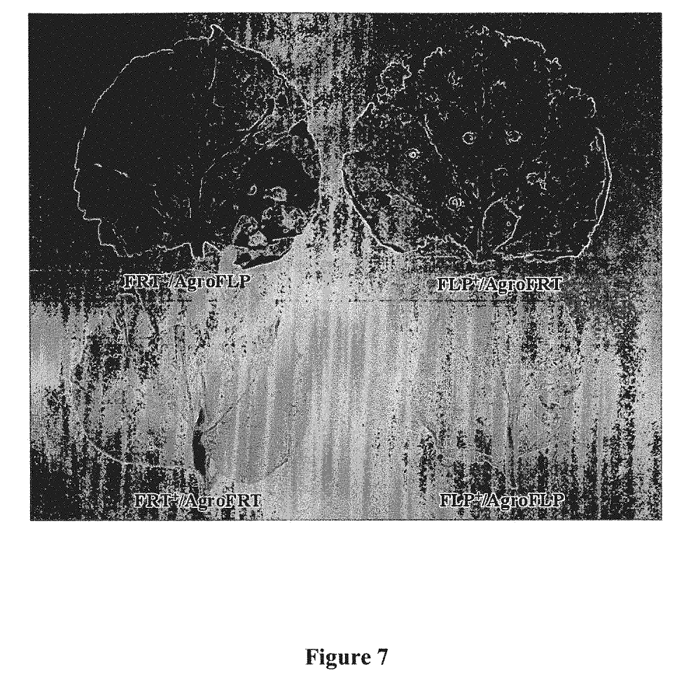

Mature leaves of each of the transgenic plants were used to test the trans-activation, i) Plants transgenic for GUS were infiltrated either with agrobacterium containing transformation vector with FLP recombinase or FRT elements. Expression of GUS was observed only in transgenic plants infiltrated with agrobacterium containing FLP recombinase (FIG. 7). When transgenic plants containing GUS gene silenced by FRT elements were infiltrated with agrobacterium carrying GUS gene flanked with FRT sequences no GUS activity was detected. ii) However, when transgenic plants expressing FLP recombinase were infiltrated with agrobacterium carrying GUS gene flanked with FRT sequences or FLP only. GUS activity was detected only when plants were agroinfiltrated with agrobacterium carrying GUS gene flanked with FRT sequences (FIG. 7).

### C. Expression of Target Gene in Transgenic Plants by Trans-Activation via Virus Infection:

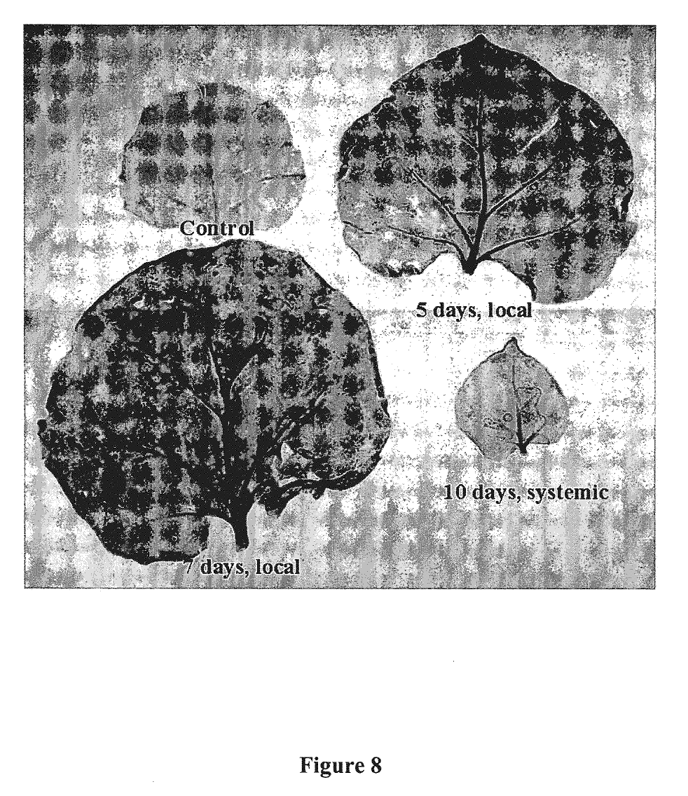

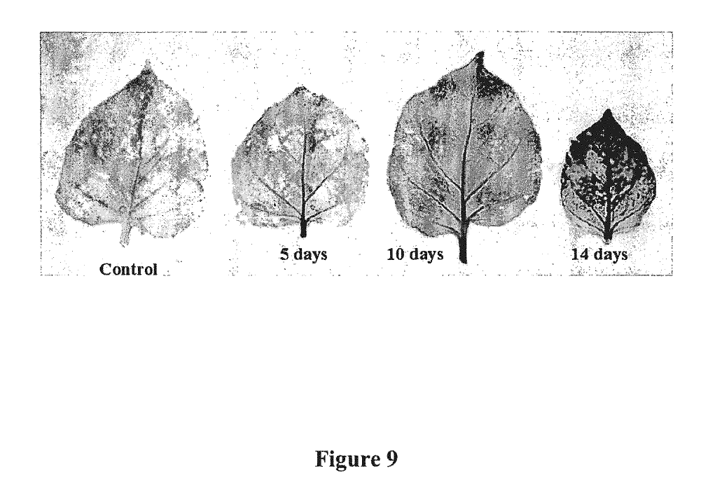

To demonstrate that the system allows for expression of target gene in stably transformed plants, transgenic N. benthamiana plants containing GUS gene silenced by staffer flanked with FRT sequences were created. These transgenic plants were inoculated with virus vector containing FLP recombinase. Infection with virus containing FLP recombinase resulted in significant GUS activity, both locally and systemically (FIGS. 8 and 9). No β-glucuronidase activity was observed in the absence of the FLP trans-activator (FIG. 8, 9,transgenic un-infected control plant). Thus, we have demonstrated that the FLP/FRT system can be used for the controlled expression of target genes in transgenic plants via trans-activation with plant virus-produced FLP recombinase.

### Example 2

### GAL4-VP16 Mediated Trans Activation of Inactive Transgenes in Cells GAL4-VP16 Trans-Activator System

DNA Constructs:

The vector pET-15 GAL4-VP16 UASmGFP5ER encodes the GAL4-VP16 gene-fusion. GAL4-VP16 was PCR amplified using oligonucleotides GAL4VP16-5′ (5′-CCAGGATCC TTAATTAATGAAGCTCCTGTCCTC-3′) and GAL4VP16-3′ (5′-ACGCGTCGACAGATCT ACCCACCGTA-3′) to introduce 5′ Pacl and a 3′ Sal I cloning sites for cloning into viral vectors based on AIMV, TMV, or CMV (FIG. 10).

GAL4 UAS Construction and Cloning:

Six copies of the GAL4 UAS was constructed by annealing the two complimentary oligonucleotides 6XGAL4 (5′-GCGGGTGACAGCCCTCCGCGGGTGACAGCCCTCC GCGGGTGACAGCCCTCCGCGGGTGACAGCCCTCCGCGGGTGACAGCCCTCCGCGGG TGACAGCCCTCCGT-3′) (SEQ ID NO:4) and Pst6XGAL4Xba (5′-CTAGACGGAGGG CTGTCACCCGCGGAGGGCTGTCACCCGCGGAGGGCTGTCACCCGCGGAGGGCTGTC ACCCGCGGAGGGCTGTCACCCGCGGAGGGCTGTCACCCGCTGCA-3′), which create a 5′ PsTI overhang and a 3′ XbaI overhang. The GAL4 fragment was cloned into pBluescript for sequence analysis.

Similarly, the minimal −46 to +8 region of the CaMV promoter was constructed by annealing the two complimentary oligonucleotides Ca.MVXba (5′-CTAGAGCGAAGACCCTT CCTCTATATAAGGAAGTTCATTTCATTTGGAGAGGACACGCTG-3′) and CaMVBam (5′-GATCCAGCGTGTCCTCTCCAAATGAAATGAACTTCCTTATATAGAGGAAGGGTCTTG CT-3′). This DNA fragment was also cloned into pBluescript for sequence analysis.

Following sequence analysis the two promoter components were cloned in tandem into pBluescript as follows. The minimal promoter was excised using BaMHi and XbaI. The 6xGAL4UAS was excised with XbaI and HindIII. The two fragments were ligated into pBluescript simultaneously and analyzed by DNA sequencing. The HindIII-BamHI fragment, encompassing both the 6xGAL4UAS and the minimal promoter sequences, was cloned into pBI121 where it replaced the 35SCaMV promoter. In the resulting vector, referred to as pBIGAL (FIG. 11), reporter (-glucuronidase)-expression is driven by the GAL4 promoter upon binding by the GAL4-VP 16 trans-activator. The β-glucuronidase encoding gene can be replaced by other target genes using the 5′ restriction site BamHI and the 3′ site SacI. IA-2 was cloned into the 5′ BamHI site and 3′ SacI sites of pBIGAL.

Viral Infection:

In vitro transcripts were synthesized using T7 RNA polymerase after KpnI linearization of TMV-based vector containing either GFP or GAL4-VP16 as follows. Approximately 10 μg of DNA was linearized with 30 units of KpnI overnight in a reaction volume of 100 μl. Four μl of the restriction digest was used to produce in vitro transcripts using the AmpliCap T7 High Yield message Maker Kit (Epicentre) according the manufacturers recommendations. Transcripts from one such reaction were used to infect 12 six-week-old Nicotiana benthamiana (transgenic or wildtype) by manually applying the transcripts dissolved in FES [sodium-pyrophosphate 1% (w/v), macaloid 1% (w/v), celite 1% (w/v), glycine 0.5 M, K2HP04 0.3 M, pH 8.5, with phosphoric acid] onto young, fully expanded leaves.

Agro-Infiltration of a Single (Reporter) Gene:

pBIGAL was transformed into Agrobacterium tumefaciens strain GLA 4404 and used to infiltrate N. benthamiana leaves that had previously been inoculated with virus expression vector containing GAL4-VP16 or GFP. Recombinant virus infection was monitored by observing GFP accumulation under UV-light. Because the GAL4-VP 16 infection progression could not be visualized definitely it was inferred that GAL4-VP 16 infection would progress similar to that of vector containing GFP progression due to the similarity in insert size of GFP and GAL4VP16. Agro-infiltration was performed when GET accumulation could be visualized. In addition, phenotypic symptoms indicative of viral infection were monitored.

An Agrobacterium culture of pBIGAL was grown to saturation in YEB containing 50 μg/ml Kanamycin (Km) at 28° C., 225 rpm. This culture was diluted 1:500 into YEB containing 50 μg/ml Km, 10 mM MES and 20 μM acetosyringone and grown at 28° C., 225 rpm to an OD₆₀₀ of 0.8. The cells were collected by centrifugation at 3000 g for 15 min and resuspended in MMA (lx MS salts, 10 mM MES pH 5.6, 20 g/l sucrose, 200 μM acetosytingone) to and OD₆₀₀ of 2.4. The MMA suspension was held at room temperature for 1-3 hrs before infiltration. Infiltration with agro was performed by gently injecting the acetosyringone-induced suspension of agrobacterium into virally infected, fully expanded, young leaves so that the agro-suspension diffused through the tissue without damaging the leaf. The leaves were left on the plants for three to four days after agro-infiltration and thereafter harvested, photographed under UV-light (to demonstrate green fluorescence of GIP in controls) and assayed for β-g lucuronidase-activity by vacuum-infiltration in GUS-buffer (0.1 m NaPO₄ pH 7.1, 0.5 mM K₃[Fe(CN₆)], 0.5 mM [Fe(CN₆)], 10 mM EDTA, 0.01% Triton X-100, 1 mg/ml x-gluc) followed by 7 hrs incubation at 37° C. The plants were de-stained in 70% ethanol over several days and photographed on a light box.

Agro-Infiltration of Two Target Genes β-Glucuronidase and IA-2:

pBIGAL carrying either β-glucuronidase or IA-2, was transformed into Agrobacterium tumefaciens strain GLA 4404 and used to infiltrate N. benthamiana leaves that had previously been inoculated with a recombinant virus vector containing GAL4-VP16 or GFP as described above. Leaves expressing the GAL4-VP16 trans-activator or the GFP control were infiltrated with either β-glucuronidase/pB1GAL or IA-2,/pBIGAL alone or with a mixture of β-glucuronidase/pBIGAL and IA-2/pBIGAL. Three to four days post-agro-infiltration the leaves were harvested and photographed. All the leaves that had been infiltrated with agrobacterium containing the β-glucuronidase/pBIGAL vector were assayed for β-glucuronidase activity as described above. Roughly half the leaves that had been infiltrated with both β-glucuronidase/pBIGAL and IA-2/pBIGAL constructs were assayed for β-glucuronidase activity, while the other half was sampled for Western blot analysis. All IA-2/pBIGAL infiltrated leaves were sampled for Western blot analysis as well. Westerns were performed as follows: Two to three one cm circular leaf-disk were taken from each leaf and frozen at −80° C. min microcentrifuge tubes. The leaf-tissue was homogenized in 100 μl 0.1 M sodium phosphate buffer, pH 7.1 using a micro-pestle attached to a Sears-brand small drill. Following homogenization, 50 μl protein SDS gel loading buffer was added to the samples and the samples were heated to 100° C. for 5 min. The samples were centrifuged at full speed in a microcentrifuge for 20 min to collect the plant debris. 10 μl of the supernatant was loaded onto a 10% polyacrylamide gel and proteins were separated by electrophoresis followed by transfer onto PVDF membrane. The membrane was blocked overnight in 1-block and probed with a primary mouse monoclonal anti-tyrosine phosphatase (IA-2ic) antibody (LAD), diluted 1:1000 in I-block, followed by a secondary horse-radish peroxidase conjugated anti mouse antibody, diluted 1:10,000 in I-block. Westerns were developed with Pierce chemiluminescent detection kit according to the manufacturer's directions.

### A. Expression of β-Glueuronidase Reporter Gene in Nicatiana Benthamiana Plant by Trans-Activation GAL4-VP 16:

To demonstrate that the inducible plant expression system described above allows for tight transcriptional control of a trans-gene the β-glucuronidase reporter gene was placed downstream of 6 repeats of the 17 by GAL4 UAS and introduced into plants transiently expressing the GAL4-VP16 activator by agro infiltration. It is shown here that the target proteins can be expressed in a fully transient manner.

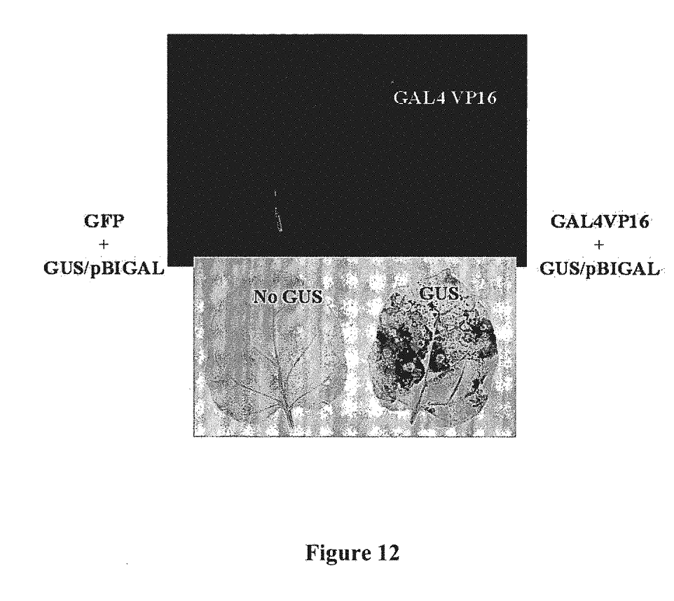

Nicotiana benthamiana plants (at 6 leaf stage) were independently inoculated with in vitro transcripts of the viral vector containing GAL4-VP16 (trans-activator) or GFP (control) gene under the control of subgenomic promoter for mRNA of viral coat protein. Approximately five to seven days post-inoculation symptoms (curling of leaves) of systemic spread of virus infection could be observed. In plants inoculated with viral vector containing GFP expression of GFP could be monitored by visualizing the green fluorescence of GFP under UV-light in upper leaves (FIG. 12).

Ten days post inoculation with in vitro transcripts of the viral vectors containing GAL4-VP16 trans-activator or GFP gene, the upper leaves were infiltrated with an agrobacterium suspension carrying pBIGAL that contain the target gene, β-glucuronidase. Two to three days post agro-infiltration the leaves were harvested and assayed for β-glucuronidase activity (FIG. 12). Strong β-glucuronidase activity was visualized in all leaves which expressed the GAL4-VP16 trans-activator and were infiltrated with agrobacterium containing the β-glucuronidase target gene, while no β-glucuronidase activity was detectable in any of the control leaves, which expressed GFP (FIG. 12). In addition, no β-glucuronidase activity was observed in leaves that expressed GAL4-V,P16 but did not receive β-glucuronidase/pBIGAL during agro-infiltration. Accordingly, the GAL4-VP16 trans-activation system allows for tight controlled expression of the desired target gene.

### B. Expression of Two Target Genes:

The trans-activation system described above can be used for expression of both single subunit as well as multi-subunit complex target proteins, including antibodies that require simultaneous expression of two target genes.

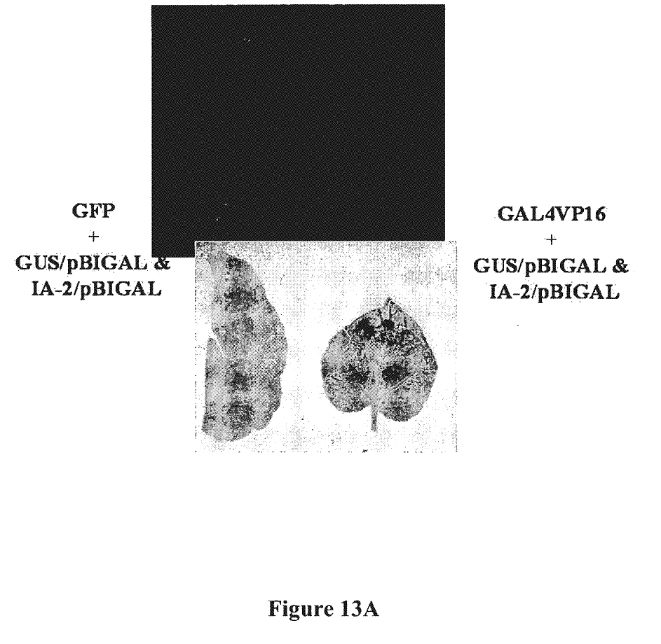

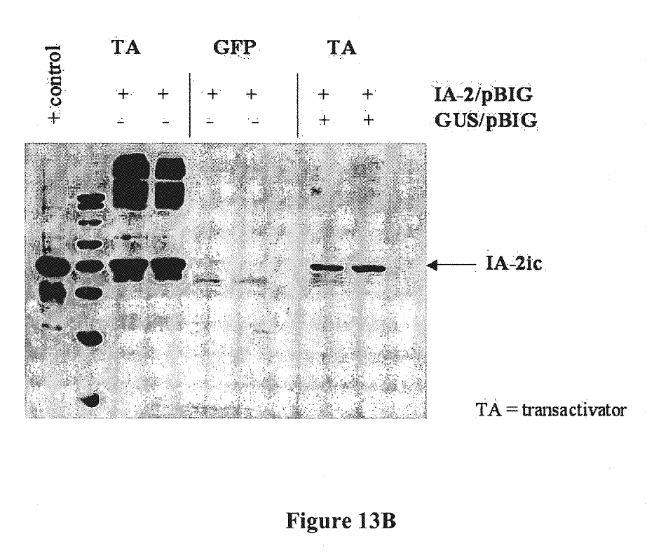

To demonstrate that the system allows for expression of more than one target gene, N. benthamiana plants were infected with a viral vector containing GAL4-VP 16 were infiltrated with agrobacterium carrying two target genes that remain inactive unless transactivated by GAL4-VP16 transcription activator (β-glucuronidase and 1A-2: Kudva Y C, Deng Y J, Govindarajan R, Abraham R S, Marietta E V, Notkins A L, David. C S. HLA-DQ8 transgenic and NOD mice recognize different epitopes within the cytoplasmic region of the tyrosine phosphatase-like molecule, IA-2. Hum Immunol. 2001 October; 62(10):1099-105.). The plants were assayed for β-gtucuronidase activity and probed for IA-2 expression at 3 days post agro-infiltration. Simultaneous expression of both β-glucuronidase and IA-2/pBIGAL (FIGS. 13A and 13B) was observed. No β-glucuronidase activity was observed in the absence of the GAL4-VP16 trans-activator (FIG. 13A), neither was there any detectable β-glucuronidase activity in plants that were infiltrated with MMA alone (not shown). Similarly, IA-2 was detected only in plants that expressed the trans-activator and not in plants expressing the GFP control (FIG. 13B). Moreover, the level of IA-2 expression was not adversely affected by the presence of a second target gene, β-glucuronidase. Thus, it has been demonstrated here that the GAL4-VP16 trans-activation system can be used for the expression of two target genes in Nicotiana benthamiana.

### C. Expression of Target Gene in Transgenic Plants by Trans-Activation:

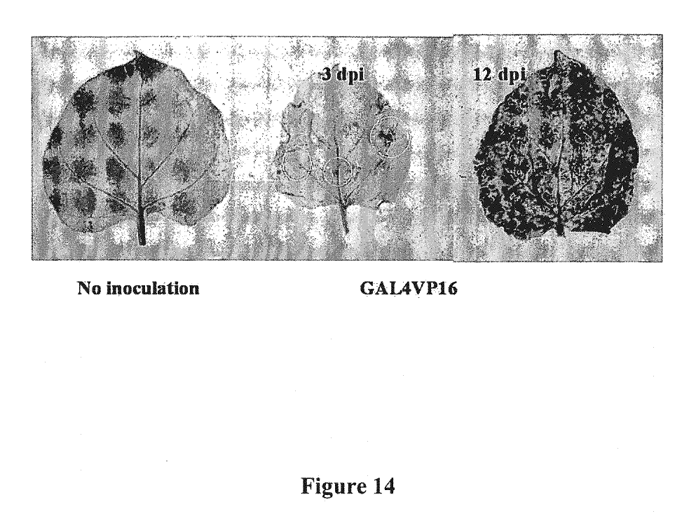

To demonstrate that the system allows for expression of target gene in stably transformed plants transgenic N. benthainiana plants containing GUS gene were created. These transgenic plants were inoculated with virus vector containing GAL4-VP16. Three and 12 (FIG. 14) days post inoculation leaves from inoculated and non-inoculated plants were assayed for β-glucuronidase activity. In leaves harvested from GAL4-VP16/D4 inoculated plants expression of β-glucuronidase was observed (FIG. 14). No β-glucuronidase activity was observed in the absence of the GAI4-VP16 trans-activator (FIG. 14), neither was there any detectable β-glucuronidase activity in plants that were infiltrated with MMA alone (not shown). Thus, it has been demonstrated here that the GAL4-VP 16 trans-activation system activates the expression of silent target genes in transgenic plants.

D. Expression of Human Anti-Tetanus Toxin Antibody in Nicotiana benthamiana Plants:

To demonstrate that the virus mediated transactivation system can be used for expression of complex multi-subunit molecules or for the expression of two polypeptide chains that assemble to form a functional protein, biologically active human anti-tetanus toxin antibody was expressed in Nicotiana benthamiana plants.

To create heavy and light chain constructs, total RNA was isolated from the 9F12 hybridoma cell line (Gigliotti et al., 1982, J Clin Invest 70, 1306-9) (American Type Culture collection number HB-8177) using RNeasy Mini Kit (Qiagen). The heavy and light chain coding regions were amplified by RT-PCR using Superscript One-Step RT-PCR, with Platinum Taq DNA polymerase (Invitrogen) introducing SfiI sites at the 3′ and 5′ ends (Hull, in preparation). The heavy and light chain PCR fragments were cloned in pBIGALSfi-GUS to create pBIGALSfi-9F12G and pBIGALSfi-9F12K, respectively. Expression activation assays, including inoculation with viral transcripts and agroinfiltration, were conducted as described in above examples. The TMV-based vector 30B having GAL4-VP16 (30B-TA) or the control virus (30B-GFPC3) infected plants were infiltrated with a mixture of agrobacteria carrying pBIGALSfi-9F12G and pBIGALSfi-9F12K. Production of both heavy and light chains was detected by Western blot analysis 2.5 days after agro-infiltration in 30B-TA infected plants but not in plants infected by 30B-GFPC3. Western blot analysis was performed using standard methods (Ausubel, et al., Current Protocols in Molecular Biology (John Wiley and Sons, New York, 1994-2003). All tissue samples were homogenized in 50 mM Tris-HCl, pH 7.5 with Complete Mini protease inhibitors (Roche Applied Science). Samples corresponding to 1.0 mg of plant tissue were loaded in each lane of a ten percent SDS-PAGE gel. The separated proteins were transferred to Immobilon-P transfer membrane (Millipore) and blocked in 0.5% I-block (Applied Biosystems). For detection of antibody heavy and light chains, a 1:2000 dilution of a rabbit anti-human lgG antibody (Jackson Immunoresearch laboratories) was used, followed by HRP-conjugated anti-rabbit antibody (Jackson Immunoresearch laboratories). The Western blots were processed using SuperSignal West Pico Chemiluminescent Substrate (Pierce). For antibody analysis, the image was taken with GeneSnap software on a GeneGnome (Syngene Bioimaging).

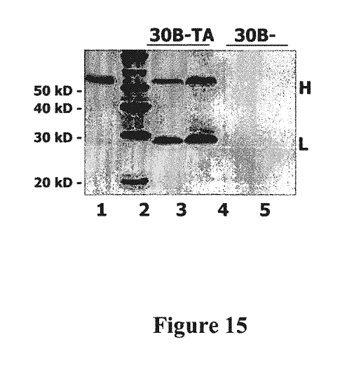

Shown in FIG. 15 is a Western blot for Detection of the heavy (H) and light (L) chains of the 9F12 antibody in plant extracts 3 days post agroinfiltration with pBIGAL-9F12G and pBIGAL-9F12K. The antibody is produced in 30B-TA infected plants (lanes 3, 4) but not in 30B-GFPC3 infected plants (lanes 5, 6). Protein samples from two independent plants were separated on SDS-PAGE and detected on membranes using rabbit polyclonal anti-GAL4 antibody (A), anti-IA-2ic monoclonal antibody (B), or rabbit polyclonal anti-human lgG antibody (C). Lane 1 in (B) and (C) contain 50 ng of IA-2ic or 200 ng whole human lgG respectively and lane 1 in (A) and lanes 2 in (B) and (C) contain MagicMark molecular weight standard (Invitrogen).

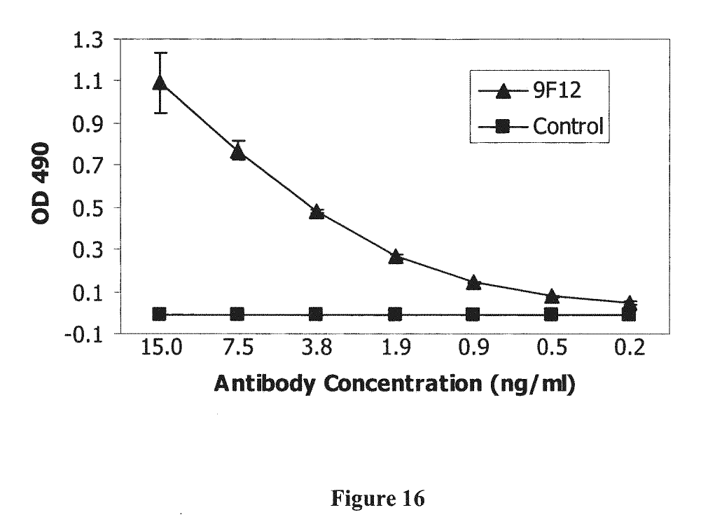

Assembled antibody was purified on a protein A column and shown to be capable of binding tetanus toxin in ELISA (Gigliotti et al., 1982, J Clin Invest 70, 1306-9), while an unrelated antibody purified from plants, did not bind tetanus toxin when used at the same concentrations in ELISA (FIG. 16), The yield of purified antibody was approximately 1 μg per gram of fresh tissue.

For Antibody Purification and ELISA, plant tissue was ground in liquid nitrogen and extracted for 1 hr at 4° C. in 50 mM Tris -HCI, pH 7.5, 0.1%717ween, 10 mM sodium diethyldithiocarbamate, 1% PPVP and Complete Mini protease inhibitors (Roche Applied Science). Plant debris was removed by two 30 min centrifugations at 75,000×g and the supernatant was filtered through a 0.45 mm filter before purification on a protein A column according to the manufacturer's protocol (Pierce). The antibody was eluted with 0.1 M glycine, pH 2.2 and neutralized with 1 M Tris base. The antibody concentration was determined by ELISA, in which serial dilutions of the antibody were plated on a Nunc Maxisorb plate and detected with a 1:2000 dilution of the rabbit anti-human IgG antibody followed by the HRP-conjugated anti-rabbit antibody. Whole human IgG (Jackson Immunoresearch laboratories) was used as a standard. About 0.5 μg of tetanus toxin (Calbiochem) in 100 μl PBS was plated as above and incubated with serial dilutions of antibodies purified from plants. Bound antibody was detected using 1:10,000 dilution of an HRP-conjugated mouse anti-human lgG antibody (Jackson Immunoresearch laboratories) and SIGM AFAST™ o-phenylenediamine dihydrochloride (Sigma).

All publications, patents and patent applications mentioned in the specification are indicative of the level of those skilled in the art to which this invention pertains. All publications, patents and patent applications are herein incorporated by reference to the same extent as if each individual publication or patent application was specifically and individually indicated to be incorporated by reference. Although the foregoing invention has been described in some detail by way of illustration and example for purposes of clarity of understanding, it will be obvious that certain changes and modifications may be practiced within the scope of the appended claims.

1. A transactivation system for producing a foreign polypeptide of interest in cells of a host plant comprising:
genetically transformed cells of the host plant having integrated in their nuclear genome, an inactive or silenced foreign nucleic acid sequence, which is capable of encoding, upon its activation, the foreign polypeptide of interest; and
a recombinant RNA viral vector capable of infecting the cells of the host plant and encoding therein a factor for activating or facilitating the expression of inactive or silenced foreign nucleic acid sequence.

2. The transactivation system of claim 1, wherein the recombinant RNA viral vector has a recombinant genomic component of a tobamovirus, an alfalfa mosaic virus, an ilarvirus, a cucumovirus or a closterovirus.

3. (canceled)

4. The transactivation system of claim 1, wherein the foreign nucleic acid sequence is integrated into a viral replicon responsive to a replication factor encoded by the recombinant RNA viral vector.

5. The transactivation system of claim 1, wherein the foreign nucleic acid sequence is integrated into a viral sequence capable of self replicating in the transformed cells of the host plant following transactivation.

6. The transactivation system of claim 1, wherein the foreign nucleic acid sequence is inactive or silenced due to a blocking sequence between the foreign nucleic acid sequence and regulatory sequences in place for driving the expression of the foreign nucleic acid sequence.

7. The transactivation system of claim 6, wherein the blocking sequence comprises a stuffer sequence flanked on each side by an FRT site in a 5′ to 3′ orientation.

8. The transactivation system of claim 1, wherein the foreign nucleic acid sequence is inactive due to the lack of a specific transcription factor activity within said cells.

9. The transactivation system of claim 1, wherein the nuclear genome has a DNA sequence to which the transcription factor can bind and activate the expression of inactive foreign nucleic acid sequence within said cells.

10-13. (canceled)

14. The transactivation system of claim 1, wherein the recombinant RNA viral vector is a heterologous viral vector.

15. A method for producing a foreign polypeptide in cells of a host plant comprising:
(a) generating a transgenic plant so that nuclear genome of cells of the transgenic plant has a transgenic DNA comprising an inactive or silenced foreign nucleic acid sequence, which is capable of encoding, upon its activation, the foreign polypeptide;
(b) infecting the transgenic plant cells with a recombinant RNA viral vector so that it replicates and transiently expresses therein a factor for activating or facilitating the expression of the inactive or silenced foreign nucleic acid sequence; and
(c) growing said plant, wherein said foreign polypeptides are produced in said cells.

16. The method of claim 15, wherein the recombinant RNA viral vector is a heterologous viral vector.

17. (canceled)

18. The method of claim 15, wherein the recombinant RNA viral vector has a recombinant genomic component of a tobamovirus, an alfalfa mosaic virus, an ilarvirus, a cucumovirus or a closterovirus.

19. The method of claim 15, wherein the foreign nucleic acid sequence is integrated into a viral replicon responsive to a replication factor encoded by the recombinant RNA viral vector.

20. The method of claim 15, wherein the foreign nucleic acid sequence is integrated into a viral sequence capable of self replicating in the transformed cells of the host plant following transactivation.

21. The method of claim 15, wherein the foreign nucleic acid sequence is inactive or silenced due to a blocking sequence between the foreign nucleic acid sequence and regulatory sequences in place for driving the expression of the foreign nucleic acid sequence.

22. The method of claim 16, wherein the blocking sequence comprises a stuffer sequence flanked on each side by an FRT site in a 5′ to 3′ orientation.

23. The method of claim 15, wherein the foreign nucleic acid sequence is inactive due to lack of activity of a transcription factor activity within said cells.

24. The method of claim 15, wherein the nuclear genome has a DNA sequence to which the transcription factor can bind and activate the expression of inactive foreign nucleic acid sequence within said cells.

25-28. (canceled)

29. A method for producing a foreign polypeptide in cells of a host plant comprising:
(a) generating transgenic plant cells so that nuclear genome of said cells has a transgenic DNA comprising an inactive or silenced foreign nucleic acid sequence, which is capable of encoding, upon its activation, the foreign polypeptide;
(b) infecting the transgenic plant cells with a recombinant RNA viral vector so that it replicates and transiently expresses therein a factor for activating or facilitating the expression of the inactive or silenced foreign nucleic acid sequence; and
(c) growing said cells in a suitable culture medium, wherein said foreign polypeptides are produced in said cells.

30. The method of claim 29, wherein the transgenic plant cells are cell suspensions or cells in a tissue selected from the group consisting of: root, shoot, flower and fruit.
